(19) United States
(12) Patent Application Publication
(10) Pub. No.: US 2021/0386337 A1
(43) Pub. Date: Dec. 16, 2021

(54) WAVEGUIDE-BASED PULSE OXIMETRY SENSOR

(71) Applicant: Covidien LP, Mansfield, MA (US)

(72) Inventor: Michael Kertser, Jerusalem (IL)

(21) Appl. No.: 16/898,952

(22) Filed: Jun. 11, 2020

(51) Int. Cl. A61B 5/1455; A61B 5/00

(52) U.S. Cl. CPC A61B 5/14552; A61B 2562/12; A61B 2562/0238; A61B 5/6832

(57) ABSTRACT

A patient monitoring sensor having a communication interface, through which the patient monitoring sensor can communicate with a monitor is provided. The patient monitoring sensor includes one or both of waveguide-based light emitter and detector, communicatively coupled to the communication interface, capable of detecting light.

### FIELD

The present disclosure relates generally to medical devices, and more particularly, to medical devices that monitor physiological parameters of a patient, such as pulse oximeters.

### BACKGROUND

In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient uses attenuation of light to determine physiological characteristics of a patient. This is used in pulse oximetry, and the devices built based upon pulse oximetry techniques. Light attenuation is also used for regional or cerebral oximetry. Oximetry may be used to measure various blood characteristics, such as the oxygen saturation of hemoglobin in blood or tissue, the volume of individual blood pulsations supplying the tissue, and/or the rate of blood pulsations corresponding to each heartbeat of a patient. The signals can lead to further physiological measurements, such as respiration rate, glucose levels or blood pressure.

One issue in such sensors relates to light emitting diodes typically used in such applications, including added bulk to the sensor, complexity, and emission of heat. Further, such sensors, based on a standard localized LED source (e.g., dual wavelength RED/IR LEDs) and detector are very sensitive to position and measure only local tissue saturation.

There is a need in the art for robust medical sensors that overcomes limitations of traditional sensors.

### SUMMARY

The techniques of this disclosure generally relate to medical devices that monitor physiological parameters of a patient, such as pulse oximeters.

In one aspect, the present disclosure provides a patient monitoring sensor having a communication interface, through which the patient monitoring sensor can communicate with a monitor. The patient monitoring sensor also includes a waveguide-based light emitter communicatively coupled to the communication interface and a detector, communicatively coupled to the communication interface, capable of detecting light. In exemplary embodiments, the waveguide-based light emitter includes a light source coupled to a waveguide. In further exemplary embodiments, the detector includes a signal pickup waveguide. In further exemplary embodiments, the waveguides and optical components are built over a soft pad.

In another aspect, the disclosure provides a patient monitoring system, having a patient monitor coupled to a patient monitoring sensor. The patient monitoring sensor includes a communication interface, through which the patient monitoring sensor can communicate with the patient monitor. The patient monitoring sensor also includes a waveguide-based light emitter communicatively coupled to the communication interface and a detector, communicatively coupled to the communication interface, capable of detecting light. In exemplary embodiments, the waveguide-based light emitter includes a light source coupled to a waveguide. In further exemplary embodiments, the detector includes a signal pickup waveguide. In further exemplary embodiments, the waveguides and optical components are built over a soft pad.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 illustrates a perspective view of an exemplary patient monitoring system including a patient monitor and a patient monitoring sensor, in accordance with an embodiment; and

FIG. 2 illustrates a perspective view of an exemplary patient monitoring sensor, in accordance with an embodiment.

### DETAILED DESCRIPTION

Traditional pulse oximeter sensor designs utilize light emitting diodes, which as typically used in such applications, includes added bulk to the sensor, complexity, and emission of heat. Further, such sensors, based on a standard localized light emitting diode (LED) source (e.g., dual wavelength RED/IR LEDs) and detector are very sensitive to position and measure only local tissue saturation.

Accordingly, the present disclosure describes a patient monitoring sensor that includes a waveguide-based light emitter communicatively coupled to the communication interface and a detector, communicatively coupled to the communication interface, capable of detecting light. In exemplary embodiments, the waveguide-based light emitter includes a light source coupled to a waveguide. In further exemplary embodiments, the detector includes a signal pickup waveguide.

In further exemplary aspects, exemplary waveguides for the source and the detector include soft waveguides configured to utilize a total internal reflection effect, where the light (e.g., IR/RED) entering the tissue is distributed over a bigger surface and penetrate the tissue only in places of contact between the waveguide material and the skin at overcritical angles, otherwise reflecting inside. In such a way, the source distributes the light injection and pickup all over the surface of the skin (e.g., on the finger) in order to sample a bigger volume inside the tissue (a volumetric measurement) for a more stable measurement, as compared with local tissue saturation measurements as in traditional pulse oximeters.

Additionally, in exemplary embodiments, the signal pickup waveguide comprises a waveguide with a matching or similar refractive index. Exemplary embodiments for waveguide material include Infrared (IR) transparent silicone, etc. Exemplary embodiments thus provide waveguides that can pick up, deliver and integrate the signal from all directions.

In further exemplary embodiments a light source includes an LED with a narrow opening angle. In exemplary embodiments, the opening angle is between about 10 and 15 degrees, though other opening angles are contemplated. In further exemplary embodiments, the opening angle is between about 5 and 20 degrees.

In further exemplary embodiments, the LED comprises a polarized or highly polarized LED; and the detector includes a polarization film (filter) over the detector. In exemplary embodiments, the shunted signal is filtered (the signal coming from the deep tissue will be non-polarized). In such exemplary embodiments, the shunted, non-scattered signal will remain, with a relatively high degree of polarization and will be filtered out.

In further exemplary embodiments, a suitable LED includes a vertical-cavity surface-emitting laser (VSCEL) diode, which can have both a narrow opening angle and high polarization.

In further exemplary embodiments, the waveguides and optical components are built over a soft pad.

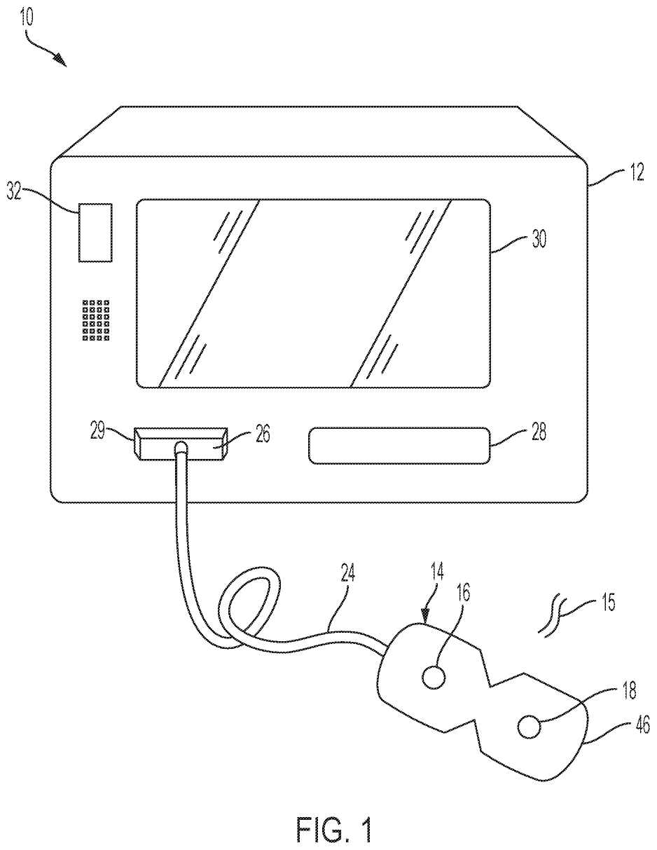

Referring now to FIG. 1, an embodiment of a patient monitoring system 10 that includes a patient monitor 12 and a sensor 14, such as a pulse oximetry sensor, to monitor physiological parameters of a patient is shown. By way of example, the sensor 14 may be a NELLCOR™, or INVOS™ sensor available from Medtronic (Boulder, Colo.), or another type of oximetry sensor. Although the depicted embodiments relate to sensors for use on a patient's fingertip, toe, or earlobe, it should be understood that, in certain embodiments, the features of the sensor 14 as provided herein may be incorporated into sensors for use on other tissue locations, such as the forehead and/or temple, the heel, stomach, chest, back, or any other appropriate measurement site.

In the embodiment of FIG. 1, the sensor 14 is a pulse oximetry sensor that includes one or more emitters 16 and one or more detectors 18. For pulse oximetry applications, the emitter 16 transmits at least two wavelengths of light (e.g., red and/or infrared (IR)) into a tissue of the patient. For other applications, the emitter 16 may transmit 3, 4, or 5 or more wavelengths of light into the tissue of a patient. The detector 18 includes a photodetector selected to receive light in the range of wavelengths emitted from the emitter 16, after the light has passed through the tissue. Additionally, the emitter 16 and the detector 18 may operate in various modes (e.g., reflectance or transmission). In exemplary embodiments, the one or both of the emitter and detector include patient-side waveguides, with a light source and photodetector removed from the patient-side by some length of the waveguide.

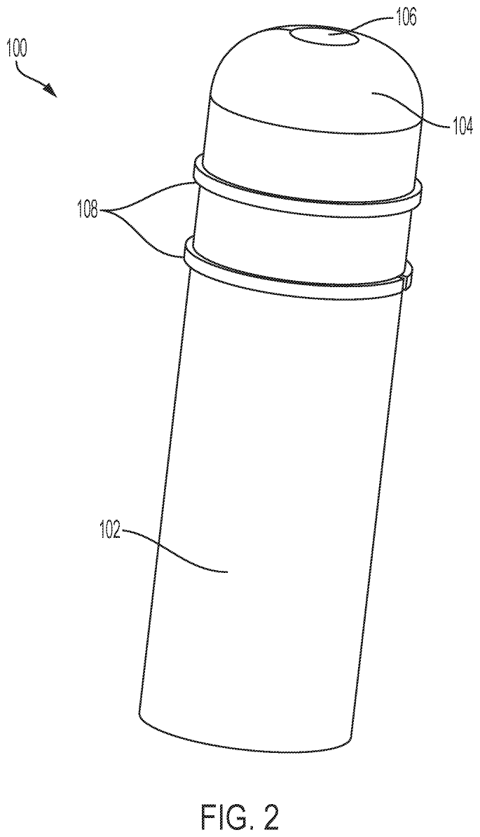

FIG. 2 illustrates a perspective view of a suitable waveguide generally at 100, which could be coupled to a light source or detector. The waveguide comprises a waveguide body 102; a rounded tip 104, configured to comfortably provide for contact with a patient's skin directly or through at least part of a bandage; a lightguide core 106; and one or more mounting ribs 108, to facilitate proper placement and orientation within a bandage relative to the skin of the patient (the patient-side of the bandage).

Referring again to FIG. 1, in certain embodiments, the sensor 14 includes sensing components in addition to, or instead of, the emitter 16 and the detector 18. For example, in one embodiment, the sensor 14 may include one or more actively powered electrodes (e.g., four electrodes) to obtain an electroencephalography signal.

The sensor 14 also includes a sensor body 46 to house or carry the components of the sensor 14. The body 46 includes a backing, or liner, bandage or pad, provided around the emitter 16 and the detector 18, as well as an adhesive layer (not shown in FIG. 1) on the patient side. The sensor 14 may be reusable (such as a durable plastic clip sensor), disposable (such as an adhesive sensor including a bandage/liner), or partially reusable and partially disposable.

In the embodiment shown, the sensor 14 is communicatively coupled to the patient monitor 12. In certain embodiments, the sensor 14 may include a wireless module configured to establish a wireless communication 15 with the patient monitor 12 using any suitable wireless standard. For example, the sensor 14 may include a transceiver that enables wireless signals to be transmitted to and received from an external device (e.g., the patient monitor 12, a charging device, etc.). The transceiver may establish wireless communication 15 with a transceiver of the patient monitor 12 using any suitable protocol. For example, the transceiver may be configured to transmit signals using one or more of the ZigBee standard, 802.15.4x standards WirelessHART standard, Bluetooth standard, IEEE 802.11x standards, or MiWi standard. Additionally, the transceiver may transmit a raw digitized detector signal, a processed digitized detector signal, and/or a calculated physiological parameter, as well as any data that may be stored in the sensor, such as data relating to wavelengths of the emitters 16, or data relating to input specification for the emitters 16, as discussed below. Additionally, or alternatively, the emitters 16 and detectors 18 of the sensor 14 may be coupled to the patient monitor 12 via a cable 24 through a plug 26 (e.g., a connector having one or more conductors) coupled to a sensor port 29 of the monitor. In certain embodiments, the sensor 14 is configured to operate in both a wireless mode and a wired mode. Accordingly, in certain embodiments, the cable 24 is removably attached to the sensor 14 such that the sensor 14 can be detached from the cable to increase the patient's range of motion while wearing the sensor 14.

The patient monitor 12 is configured to calculate physiological parameters of the patient relating to the physiological signal received from the sensor 14. For example, the patient monitor 12 may include a processor configured to calculate the patient's arterial blood oxygen saturation, tissue oxygen saturation, pulse rate, respiration rate, blood pressure, blood pressure characteristic measure, autoregulation status, brain activity, and/or any other suitable physiological characteristics. Additionally, the patient monitor 12 may include a monitor display 30 configured to display information regarding the physiological parameters, information about the system (e.g., instructions for disinfecting and/or charging the sensor 14), and/or alarm indications. The patient monitor 12 may include various input components 32, such as knobs, switches, keys and keypads, buttons, etc., to provide for operation and configuration of the patient monitor 12. The patient monitor 12 may also display information related to alarms, monitor settings, and/or signal quality via one or more indicator lights and/or one or more speakers or audible indicators. The patient monitor 12 may also include an upgrade slot 28, in which additional modules can be inserted so that the patient monitor 12 can measure and display additional physiological parameters.

Because the sensor 14 may be configured to operate in a wireless mode and, in certain embodiments, may not receive power from the patient monitor 12 while operating in the wireless mode, the sensor 14 may include a battery to provide power to the components of the sensor 14 (e.g., the emitter 16 and the detector 18). In certain embodiments, the battery may be a rechargeable battery such as, for example, a lithium ion, lithium polymer, nickel-metal hydride, or nickel-cadmium battery. However, any suitable power source may be utilized, such as, one or more capacitors and/or an energy harvesting power supply (e.g., a motion generated energy harvesting device, thermoelectric generated energy harvesting device, or similar devices).

As noted above, in an embodiment, the patient monitor 12 is a pulse oximetry monitor and the sensor 14 is a pulse oximetry sensor. The sensor 14 may be placed at a site on a patient with pulsatile arterial flow, typically a fingertip, toe, forehead or earlobe, or in the case of a neonate, across a foot. Additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. The patient monitoring system 10 may include sensors 14 at multiple locations. The emitter 16 emits light which passes through the blood perfused tissue, and the detector 18 photoelectrically senses the amount of light reflected or transmitted by the tissue. The patient monitoring system 10 measures the intensity of light that is received at the detector 18 as a function of time.

A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The amount of light detected or absorbed may then be used to calculate any of a number of physiological parameters, including oxygen saturation (the saturation of oxygen in pulsatile blood, SpO2), an amount of a blood constituent (e.g., oxyhemoglobin), as well as a physiological rate (e.g., pulse rate or respiration rate) and when each individual pulse or breath occurs. For SpO2, red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood, such as from empirical data that may be indexed by values of a ratio, a lookup table, and/or from curve fitting and/or other interpolative techniques.

One or more specific embodiments of the present techniques will be described below. In an effort to provide a concise description of these embodiments, not all features of an actual implementation are described in the specification. It should be appreciated that in the development of any such actual implementation, numerous implementation-specific decisions must be made, which may vary from one implementation to another.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A patient monitoring sensor, comprising
a communication interface, through which the patient monitoring sensor can communicate with a monitor;
a light-emitting source, communicatively coupled to the communication interface, the light emitting source including a patient-side waveguide configured to direct light therethrough; and
a detector, communicatively coupled to the communication interface, capable of detecting light.

2. The patient monitoring sensor of claim 1, wherein the detector includes a patient-side waveguide configured to collect light.

3. The patient monitoring sensor of claim 1, wherein the light emitting source comprises a light emitting diode (LED) with a narrow opening angle of between about 10 and 15 degrees.

4. The patient monitoring sensor of claim 3, wherein the light emitting source comprises an LED emitting polarized light.

5. The patient monitoring sensor of claim 4, wherein the light emitting source comprises a vertical-cavity surface-emitting laser (VSCEL) diode.

6. The patient monitoring sensor of claim 4, wherein the detector includes a patient-side waveguide configured to collect light and wherein the detector is configured with a polarized filter configured to filter a shunted signal.

7. The patient monitoring sensor of claim 6, wherein the detector is configured to collect the shunted, non-scattered signal provided from patient tissue from the light emitting source waveguide.

8. The patient monitoring sensor of claim 1, wherein at least one waveguide included with one or both of the source and the detector comprises a waveguide body, an optical core, and a rounded patient-side tip.

9. The patient monitoring sensor of claim 8, wherein the at least one waveguide includes one or more mounting surfaces configured to secure the waveguide in a bandage or pad providing a patient-side orientation that delivers light or detects light from the patient side of the bandage or pad.

10. The patient monitoring sensor of claim 1, wherein the sensor is configured such that the source distributes a light injection to a surface of the skin and such that the detector picks up light from a surface of the skin to sample a volume inside the skin tissue as a volumetric measurement.

11. A method for making a patient monitoring system, comprising:
providing a communication interface, through which the patient monitoring sensor can communicate with a monitor;
coupling a light-emitting source communicatively to the communication interface, the light emitting source including a patient-side waveguide configured to direct light therethrough; and
coupling a detector capable of detecting light communicatively to the communication interface.

12. The method of claim 11, wherein the detector includes a patient-side waveguide configured to collect light.

13. The method of claim 11, wherein the light emitting source comprises a light emitting diode (LED) with a narrow opening angle of between about 10 and 15 degrees.

14. The method of claim 13, wherein the light emitting source comprises an LED emitting polarized light.

15. The method of claim 14, wherein the light emitting source comprises a vertical-cavity surface-emitting laser (VSCEL) diode.

16. The method of claim 14, wherein the detector includes a patient-side waveguide configured to collect light and wherein the detector is configured with a polarized filter configured to filter a shunted signal.

17. The method of claim 16, further comprising collecting, via the detector, the shunted, non-scattered signal provided from patient tissue from the light emitting source waveguide.

18. The method of claim 11, wherein at least one waveguide included with one or both of the source and the detector comprises a waveguide body, an optical core, and a rounded patient-side tip.

19. The method of claim 18, further comprising mounting the at least one waveguide via one or more mounting surfaces configured to secure the waveguide in a bandage or pad providing a patient-side orientation that delivers light or detects light from the patient side of the bandage or pad.

20. The method of claim 11, further comprising distributing via the source waveguide a light injection to a surface of the skin and such that the detector picks up light from a surface of the skin to sample a volume inside the skin tissue as a volumetric measurement.
